(19) United States
(12) Patent Application Publication
(10) Pub. No.: US 2009/0240123 A1
(43) Pub. Date: Sep. 24, 2009

(54) DETERMINING RELATIVE BLOOD HEMATOCRIT LEVEL USING AN AUTOMATED INTEGRATED FLUID DELIVERY AND BLOOD ACCESS DEVICE

(76) Inventors: Wayne SIEBRECHT, Golden, CO (US); Jill KLOMHAUS, Longmont, CO (US); Julie REICHERT, Aurora, CO (US)

Correspondence Address: MORRISON & FOERSTER LLP, 755 PAGE MILL RD, PALO ALTO, CA, 94304-1018, US

(21) Appl. No.: 12/050,823

(22) Filed: Mar. 18, 2008

(51) Int. Cl. A61B 5/1455

(52) U.S. Cl. 600/310

(57) ABSTRACT

Devices and methods for determining the fluid characteristics of a blood sample may include the assessment of a pressure waveform of a fluid sample having a pre-determined volume passed through or out of a flow restrictor. The interface between the blood and other fluids in the tubing line of the blood monitoring system may also be assessed. These assessments may be used alone or in combination to generate estimates of other fluid characteristics, such as the hematocrit of a blood sample. This information may be used for the real-time determination of change of hematocrit levels in a patient's blood while the patient is being transfused with intravenous fluids or other vascular products, or to provide adjustment factors for other blood assays affected by the hematocrit or other blood parameters.

### FIELD OF THE INVENTION

The embodiments disclosed herein generally relate to the field of automated blood parameter measurement and access to a patient's blood, and including a system and method for real-time determination of change of hematocrit levels in a patient's blood while the patient is being transfused with intravenous fluids.

### BACKGROUND OF THE INVENTION

Patients who have depleted fluid levels and/or blood volume from trauma, surgery or other disease may require periodic testing of hematocrit levels to assess hemodynamic status. Sometimes, an infusion of intravenous (IV) fluids to help correct the resultant physical trauma. When large volumes of IV fluids are used to stabilize the patient, transfusion with a red blood cell (RBC) source may be required to prevent excessive hemodilution (reduction in concentration of RBC) in the patient.

Physicians typically base the decision to transfuse blood and the amount of blood to transfuse on several factors, such as the most recent measured hematocrit level of the patient, size of the patient, the cardiovascular status of the patient, the coagulation state of the patient, and amount of crystalloid or colloid fluids that need to be to be administered, for example. The hematocrit is the ratio of the volume of red blood cells to the total volume of a given fluid sample. The timing and rate at which to infuse blood into a patient may be based upon the judgment of the attending medical staff. Such a trial-and-error approach may result in a variability of patient hematocrit values, since blood is traditionally transfused via units when the hematocrit falls below a certain threshold level.

The hematocrit also has an effect on certain other blood parameters. The measurements of blood analytes may be subject to variations relating to the hematocrit because the analyte may be located in the plasma fraction of the blood sample. To address the potential effect of the cellular fraction of the blood sample, the red and/or white blood cell fractions are sometimes removed prior to performing a blood assay.

### BRIEF SUMMARY OF THE INVENTION

Devices and methods for determining the fluid characteristics of a blood sample may include the assessment of a pressure waveform of a fluid sample having a pre-determined volume passed through or out of a flow restrictor. The interface between the blood and other fluids in the tubing line of the blood monitoring system may also be assessed. These assessments may be used alone or in combination to generate estimates of other fluid characteristics, such as the hematocrit of a blood sample. This information may be used for the real-time determination of change of hematocrit levels in a patient's blood while the patient is being transfused with intravenous fluids or other vascular products, or to provide adjustment factors for other blood assays affected by the hematocrit or other blood parameters.

In one embodiment, a system for assessing a blood parameter is provided, comprising a fluid channel, a sensor system configured to detect a beginning and an end of a blood/non-blood interface in the fluid channel, and a sensor processor configured to determine a difference factor between the beginning and the end of the blood interface. In some embodiments, the sensor system comprises at least one optical sensor, which may or may not be a movable optical sensor. The difference factor may be a time-based difference factor and/or a distance-based difference factor. The sensor processor may be further configured to generate a blood parameter using the difference factor. In some embodiments, the blood parameter is selected from a hematocrit, a hemoglobin, a blood viscosity, and a blood density. The fluid channel may be a tubing line or a fluid reservoir or cavity. In some instances, the system of claim may further comprise a test medium advancement mechanism comprising a motor, a blood sample dispenser, and a fluid pump. The system may also further comprise a vascular access device attachable to the fluid channel and/or a plurality test mediums or substrates, which may be of the single-use type.

In another embodiment, a system for assessing a blood parameter is provided, comprising a flow structure, a sensor system configured to detect a pressure waveform of blood passing through the flow structure, and a sensor processor configured to generate a blood parameter based upon the pressure waveform and the quantity of blood associated with the waveform. The flow structure may be an inline flow restrictor, or an open orifice, for example. The blood parameter may be selected from a hematocrit, a hemoglobin, a blood viscosity, and a blood density. The system may also further comprise a test medium advancement mechanism comprising a motor, a blood sample dispenser, and a fluid pump. The system may also further comprise a vascular access device attachable to the fluid channel, and/or a plurality test mediums or substrates, which may be of the single-use type.

In one embodiment, a method for assessing a blood characteristic is provided, comprising passing a volume of blood through a flow channel comprising having at least one flow channel structural characteristic, measuring the pressure waveform of the volume of blood, and determining a fluid characteristic of the volume of blood based upon the pressure waveform. In some embodiments, determining the fluid characteristic of the volume of blood may be further based upon at least one flow channel structural characteristic of the flow channel. In some instances, the flow channel may comprise an inline flow restrictor, or an open orifice.

In another embodiment, a method for assessing a blood characteristic is provided, comprising withdrawing a blood sample from a patient and into a channel filled with a fluid, assessing an interface between the blood sample and the fluid, and generating a fluid characteristic based upon the interface. In some embodiments, the interface between the fluid and the blood sample may be selected from a group consisting of a temperature difference, a surface-to-surface interface and an optical difference. In some embodiments, assessing the interface between the blood sample and the fluid may comprise identifying a beginning interface point between the blood sample and the fluid, and identifying an ending interface point between the blood sample and the fluid. In some examples, generating the fluid characteristic based upon the interface may comprise generating the fluid characteristic based upon a difference between the beginning interface and the ending interface. The difference may be a time-based difference or a distance-based difference.

In another embodiment, a method for performing blood monitoring is provided, comprising obtaining a blood sample from a patient using an automated blood sampling assembly, determining a non-reactive parameter and a reactive parameter of the blood sample, and adjusting the reactive parameter based upon the non-reactive parameter. The method may further comprise returning at least a portion of the blood sample to the patient using the automated blood sampling assembly. The non-reactive parameter may be a hematocrit, or may be a mechanical parameter. Examples of a mechanical parameter include a blood viscosity or blood density. In some embodiments, the reactive parameter is blood glucose.

In another embodiment, a method for performing blood monitoring is provided, comprising obtaining a blood sample from a patient using an automated blood sampling assembly, assessing a mechanical property of the blood sample, and adjusting the automated blood sampling assembly based upon the mechanical property. In one embodiment, the method further comprises converting the mechanical property into a hematocrit-related property. In some embodiments, adjusting the automated blood sampling assembly may comprises setting a test substrate adjustment factor or setting a blood sample dispensing volume, for example.

### BRIEF DESCRIPTION OF THE DRAWINGS

These and other features and advantages will be appreciated, as they become better understood by reference to the following detailed description when considered in connection with the accompanying drawings, wherein:

FIG. 1 is a functional layout of an automated blood access device and fluid infusion delivery system;

FIG. 2 is a graph of pressure waveforms based upon dispensing blood samples having different hematocrits from an orifice of a known size;

FIG. 3 is a graph of various pressure waveforms and their relative hematocrit readings during blood withdrawal through tubing having a small internal diameter;

FIGS. 4A to 4D are schematic cross-sectional views of the blood/fluid interface with different hematocrits;

FIGS. 5A to 5C are schematic cross-sectional views of various blood sensor embodiments;

FIG. 6 is a schematic cross-sectional view of another embodiment of a blood sensor; and

FIG. 7 is a schematic cross-sectional view of a disrupted blood/fluid interface.

### DETAILED DESCRIPTION OF THE INVENTION

In massively transfused patients, it is sometimes desirable to maintain blood volume using whole blood or packed red blood cells with intravenous fluids to maintain a patient's hematocrit. Conventionally, hematocrit measurements are performed using laboratory devices that require a separate blood sample and thus cannot be taken in “real-time”. Also, hematocrit measurement may also be used to make adjustments to other measured blood parameters that may be affected by the size of the cellular fraction of the blood sample.

Accordingly, there is a need for periodic, real-time monitoring of hematocrit levels in a patient during infusion of intravenous fluids, and to provide accurate measurement of other blood parameters. There is also a need for an integrated automated system that combines intravenous fluid infusion function with real-time bedside hematocrit monitoring.

Some of the embodiments described herein are directed towards a method and system for monitoring change in a patient's blood hematocrit levels. Further, some embodiments are directed towards a system and method for measuring the change in a patient's blood hematocrit level while the patient is being transfused with intravenous (IV) fluids. Still further, some embodiments are directed towards a system and method for determining a patient's blood hematocrit levels by using the time variation of pressure change that occurs when pressurized blood, contained in a closed loop system, is relieved through an orifice of known size.

Reference will now be made to certain embodiments disclosed herein. Additional features and advantages will become apparent to those skilled in the art upon consideration of the following detailed description of specific embodiments. It will thus be understood that no limitation of the scope of the invention is thereby intended. The embodiments described herein are not a general disavowal of any one specific embodiment or used to limit the claims beyond the meaning of the terms used therein.

In one embodiment, a system and method for measuring hematocrit levels in blood is provided, where the hematocrit is the ratio of the volume of red blood cells to the total volume of a given fluid sample. In another embodiment, the system and method for monitoring change in a patient's blood hematocrit levels is performed on a real-time basis. In another embodiment, a system and method for determining a patient's blood hematocrit levels comprises using the time variation of pressure change that occurs when pressurized blood, contained in a closed loop system, is relieved through an orifice of known size, or passed through a flow restrictor with know resistance characteristics.

In some embodiments, pressure wave characteristics are assessed while moving a predetermined volume of blood using a pump against a closed fluid pathway. The shape and/or amplitude of the pressure wave may be used to assess or augment the hematocrit related blood parameter. In some embodiments, assessment of the fluid interface between blood and a non-blood fluid is assessed to determine the hematocrit, or to augment the hematocrit or other blood parameter readings that may vary with the hematocrit, or with other blood volume parameters such as the hemoglobin level.

In one embodiment, an automated blood monitoring system is provided, comprising an automated intravenous (IV) fluids transfusion and blood access device integrated with a blood dispensing orifice of known size. During baseline operation, the system delivers IV fluids to the patient. During hematocrit level testing or other blood parameter testing, the system may halt or interrupt fluid delivery. Subsequently, patient blood is automatically drawn and dispensed through the dispensing orifice. The relative pressure change that occurs during blood withdrawal and dispensing via the dispensing orifice is monitored. In one embodiment, this pressure change is used to estimate the hematocrit level of blood.

In another embodiment, an automated intravenous (IV) fluids transfusion and blood access device utilizes a length of tubing having a small internal diameter that is used as a restrictor during blood withdrawal and re-infusion. The restrictor may be configured to generate a pressure change that is monitored and used to estimate the hematocrit level of blood.

In one embodiment, an automated intravenous (IV) fluids transfusion and blood access device is provided, comprising an IV drip assembly and an automated blood monitoring system where blood is automatically drawn from the patient for testing of the hematocrit and/or other blood related parameters. In some embodiments, the unused blood that was withdrawn from the patient is re-infused into the patient. This may reduce the degree of blood loss relating to the blood work performed. In some embodiments, the blood monitoring system may be a closed loop system.

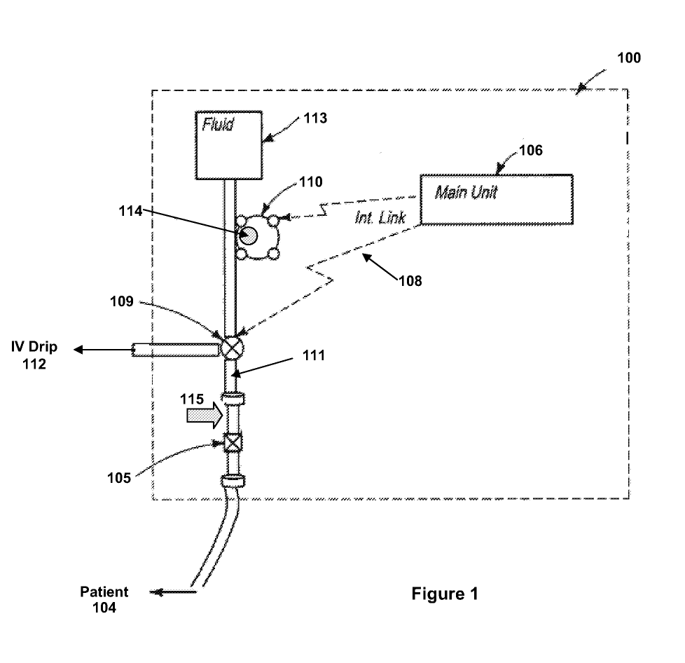

FIG. 1 is a functional layout of an automated blood access device and fluid infusion delivery system. As shown in FIG. 1, in one embodiment, the automated blood access device and fluid infusion delivery system 100 is integrated with blood dispensing assembly 105. The system 100 is connected to a catheter or other type of vascular access device to withdraw blood from patient 104 for dispensing blood samples via the dispensing assembly 105. A main microprocessor control unit 106 may be programmed to manage, via communication links 108 (that may be wired or wireless), the functioning of an infusion pump 110, and one or more stopcocks 109 or control valves for controlling the flow inside line 111 and flow of infusion fluids from one or more fluid sources. In some embodiments, at least one fluid source comprises an IV drip 112. The automated dispensing system 100 may be used to withdraw a blood sample of known volume from line 111 for testing.

In one embodiment, the infusion pump 110 is a volumetric pump, such as, but not limited to a syringe pump. In other embodiments, other types of pumps, such as, but not limited to peristaltic pumps or piston pumps can be used. In one embodiment, the infusion pump 110 is used to control the flow in the fluid delivery line from one or more fluid container 113 as well as to control the flow in line 111 used for drawing blood samples for provisioning through dispensing assembly 105.

In one embodiment, a blood sensor 115 is used to establish whether undiluted blood has reached the tube segment located above dispensing assembly 105. In one embodiment, a blood sensor 115 may be an optical sensor, wherein the sensor operates by exposing the contents of the tube to a light, receiving a transmitted or reflected signal back from the light exposure, and measuring the signal to determine if it is indicative of the presence of blood. However, in alternate embodiments, the sensor 115 may also be based on temperature, pressure or any other variable that one of ordinary skill in the art would appreciate can be used to indicate the presence or absence of blood.

In one embodiment, a pressure sensing apparatus 114 is connected to the volumetric pump. In one embodiment, the pressure sensing apparatus 114 comprises an integrated circuit connected in parallel to a load cell retrofitted on or proximal to the working end of pump mechanism such as the plunger of the pump. The load cell may also be in communication with the fluid pathway adjacent to the pump. The load cell measures the force on the plunger. In some embodiments, the pressure sensing apparatus may comprise a MEMs-type or a piezo-electric based pressure sensor, for example. In operation, the integrated circuit receives input from pump mechanism. The pressure applied by the push and pull movement of plunger is input into the load cell, which translates the pressure applied into an analog pressure value. The analog pressure value is then transferred to the integrated circuit, where it is translated into a digital value. The converted digital pressure signals are then transferred to the main unit 106.

The aforementioned automated blood access device and its operation are disclosed in U.S. patent application Ser. Nos 11/048,108; 11/288,031; and 11/386,078, which are hereby incorporated by reference in their entirety.

During baseline operation, stopcock 109 enables infusion fluid from the IV drip 112 to flow freely into patient 104 while simultaneously blocking the line coming from fluid bag 113. When performing automated blood sampling and blood hematocrit level measurements, main unit 106 directs stopcock 109 to block incoming infusions from IV drip 112 and to open the line from fluid bag 113 to patient 104. Fluid bag 113 may comprise a purging fluid such as saline solution, or other type of intravenous solution. Once the external infusions are interrupted, the pump 110 withdraws blood from the patient 104. The blood is drawn along the tube 111 until the remaining infusion volume and the initially diluted blood volume passes dispensing assembly 105.

When undiluted blood reaches blood sensor 115 located just above the assembly 105, the main unit 106 actuates a valve that isolates the patient from the pump, reverses the motion of the pump (push back) against this closed valve and then opens a separate (or same valve if multi-positioned) such that the pump forces a sample of undiluted blood out of the dispensing assembly 105. Once dispensing of the sample has occurred, the dispenser is closed and the patient isolation valve is opened and the remaining blood drawn to obtain the sample is pumped back into the patient 104. The pressure sensing apparatus 114 may be used to measure the change in pressure when the pressure in line 111 during this ‘push-back’ and is relieved through the dispensing assembly 105. In some embodiments, both of these pressures may change in magnitude/signal shape with different hematocrits. The relationship between the pressure waveforms and the hematocrit levels is discussed in greater detail below. The assembly 105 is open to atmospheric pressure when dispensing a known volume of blood using the volume pump 110.

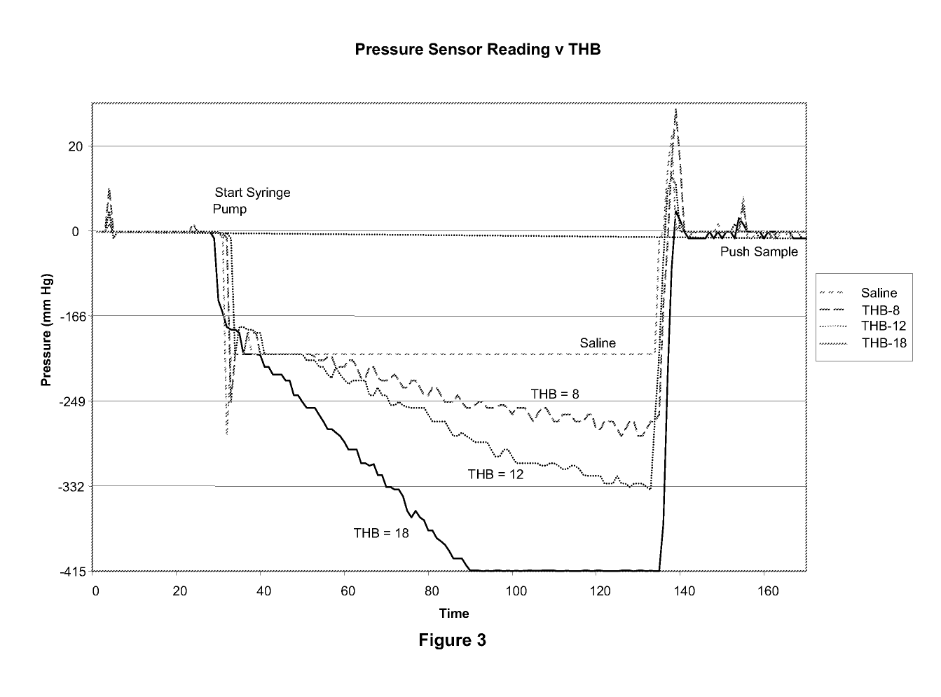

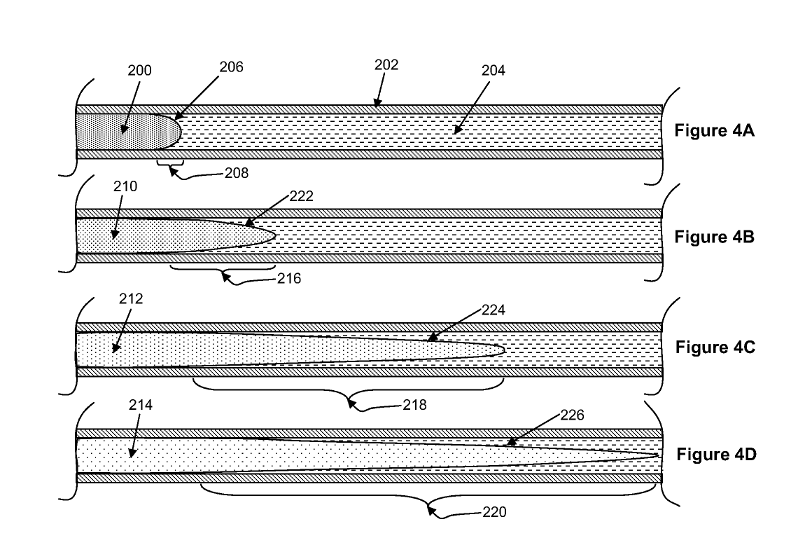

As illustrated in FIG. 4A, for example, it is believed that as blood 200 is drawn into the tubing 202 of a system filled with a fluid 204, the interface 206 between the blood 200 and the fluid 204 may vary in its morphology depending upon the hematocrit or hemoglobin level. The fluid 204 may be any of a variety of fluids, including but not limited to distilled water, D5 water, D5 half-normal saline, normal saline, lactated Ringer's solution, D5 in lactated Ringer's solution, Dextran 60 or 70, Hetastarch in water or saline, and the like. The fluid may also include one or more other agents, including but not limited to calcium gluconate, potassium chloride, sodium bicarbonate, magnesium sulfate, multi-vitamins, albumin, and the like.

Referring still to FIG. 4A, it is believed that at higher hematocrit levels, the transition or interface 206 between the blood 200 and the fluid 204 has a relatively shorter length 208 along the axis of flow through the tubing 202. Referring to FIGS. 4B to 4D, in contrast, as the hematocrit of the blood 210, 212 and 214 decreases, the lengths 216, 218 and 220 of the interfaces 222, 224 and 226 may begin to increase. This measurement of the interface length may be used as a factor in assessing or estimating the hematocrit. The assessment of this blood parameter may be used alone or in conjunction to assess the hematocrit or to produce a hematocrit-related correction factor for other blood parameter testing or physiological testing. For example, blood velocity and/or blood density may be estimated from the hematocrit and blood temperature. In a further example, measurement of the hematocrit in conjunction with temperature and Swan-Ganz catheter measurements may be used to determine the average blood velocity or average blood pressure.

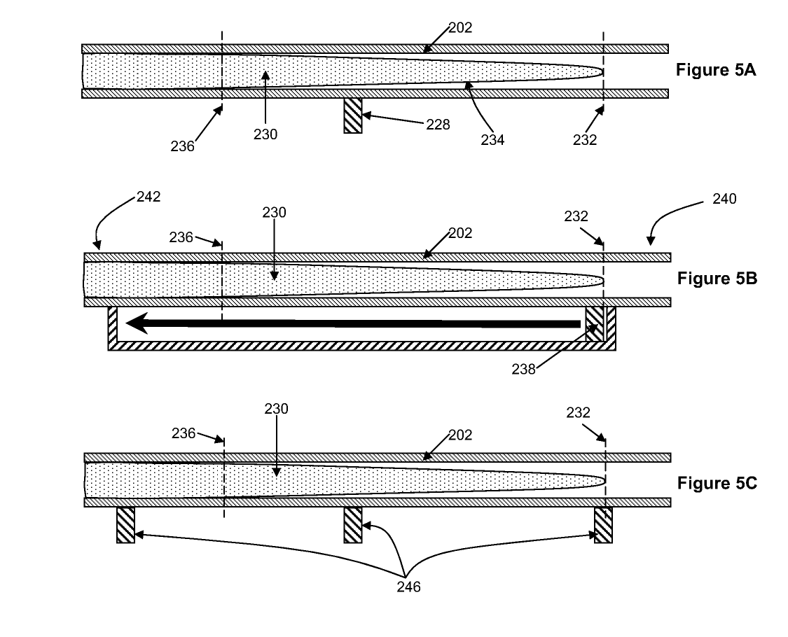

The assessment of the blood/fluid interface may be performed using one or more optical sensors. In FIG. 5A, for example, a single optical sensor 228 may be used to assess the hematocrit of the blood 230. In this particular example, the optical sensor 228 may provide a continuous or rate sampled measurement of the interaction between a light source and the contents of the tubing 202. In some embodiments, the data may be average or a trailing number of samples to augment the reliability of the sensor measurements, however, any of a variety of other error correction algorithms may be used in conjunction with the optical sensor.

Based upon changes in the continuous or sample data stream, the start 232 of the blood/fluid interface 234 may be determined by the onset of a change in the sensor signal as the blood 230 enters the visual field of the sensor 228. In some embodiments, the end 236 of the blood fluid interface 234 may also be determined by the lack of significant change in the sensor signal. In the embodiments comprising a single fixed optical sensor 228, the distance between the start 232 and the end 236 of the blood/fluid interface 232 may be determined by the time difference between the detection of the start 232 and the end 236 of the blood/fluid interface 232 and the flow velocity through the tubing 202. The flow velocity may be determined based upon the pump characteristics and the tubing dimensions. Alternatively, the estimation of the hematocrit may be based upon the time difference.

In other embodiments, the optical sensor may be configured to move. In FIG. 5B, for example, a movable sensor 238 may be positioned at the upstream end 240 of the tubing 202. When the blood/fluid interface 234 is detected by the movable sensor 238, blood flow through the tubing 202 may be suspended and the movable sensor 238 is used to scan the blood/fluid interface 234 as the movable sensor 238 is moved toward the downstream end 242 of the tubing 202. Once the end 236 of the blood/fluid interface is identified, the distance between the start 232 and the end 236 of the of the blood/fluid interface 232 may be determined. The movable sensor 238 may optionally include a housing 244 or rail member provide a movement pathway for the sensor 238.

In alternate embodiments, a plurality of fixed sensors placed along the tubing may also be used to assess the blood/fluid interface. In FIG. 5C, for example, a plurality of optical sensors 246 along the tubing 202 may be used instead of a movable sensor. In these and other embodiments, the number of optical sensors may be increased to improve the accuracy and/or reliability of the measurements.

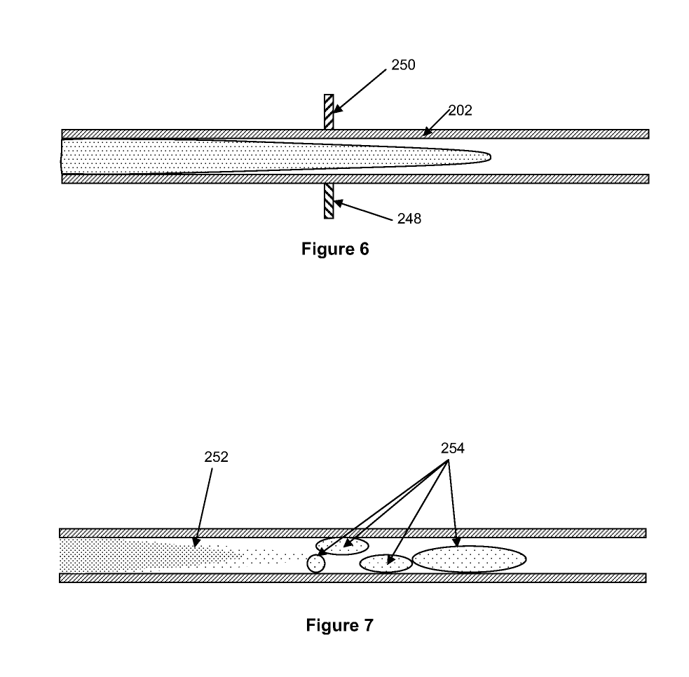

In some embodiments, one or more light sources are used to provide expand the measurement range of the optical sensor(s). The light sources may be configured along with the optical sensor(s) to perform reflective and/or transmission optical analysis of the blood and/or fluid in the tubing 200. FIG. 6, for example, depicts one embodiment comprising an optical sensor 248 and a light source 250 generally located on opposite positions relative to the tubing 202 to perform transmission optical measurements. In other embodiments, the light source and the optical sensor(s) may be configured to perform reflective optical analysis. In some of these embodiments, the light source and the sensor may have co-axial positions or may be offset by an angular measurement, for example, of about 0 degrees to about 180 degrees, sometimes about 5 degrees to about 90 degrees, and other times about 30 degrees to about 45 degrees.

The tubing 202 used to may be flexible or rigid, colored or uncolored, with a reflective or non-reflective surface, and may be optically clear or opaque. In one specific embodiment, rigid, uncolored, non-surface reflective, optically clear tubing is used in the blood monitoring system to perform the optical measurements. In some embodiments, the inner diameter or transverse dimension of the tubing may be in the range of about 0.5 mm to about 1.4 mm, sometimes about 1.5 mm to about 3.2 mm, and other times about 6 mm to about 10 mm. In some embodiments, the tubing material used in conjunction with the optical sensor assembly is similar to the other tubing material of the blood monitoring system. In other embodiments, the tubing material may be different. In some embodiments, the optical sensor assembly may be attached to other components of the blood monitoring system using any of a variety of connectors or connector structures on the other components. In some embodiments, the minimum distance between an optical sensor of the blood monitoring system and a connector interface is about 150 mm to about 300 cm, other times about 38 cm to about 76 cm and other times about 300 cm to about 500 cm or more. In some embodiments, a connector site may be associated with turbulent flow, and a minimum distance between an optical sensor and a connector may be beneficial to reduce error in the optical measurements. In some embodiments, a minimum distance is also provided between an optical sensor and any bend or turn in the fluid pathway. These and other features in the fluid pathway may cause blurring, slippage, or breakup of the blood/fluid interface. FIG. 7 depicts one example of a blurred interface or wavefront. In this particular example, the blood/fluid interface 252 may be indistinct due to blurring, which may make the detection of the interface 252 less accurate. Also, turbulent flow may break up the interface, possibly resulting in satellite blood particles 254 that may be detected by the optical sensor(s) as an irregular pattern. In some embodiments, the signal processing system for the optical sensor assembly may be configured to detect the irregularity and reject the hematocrit value calculated from irregular pattern. In some embodiments, the blood and fluid may be dumped into the waste receptacle of the system or returned to the patient, and the hematocrit detection system may be reinitiated.

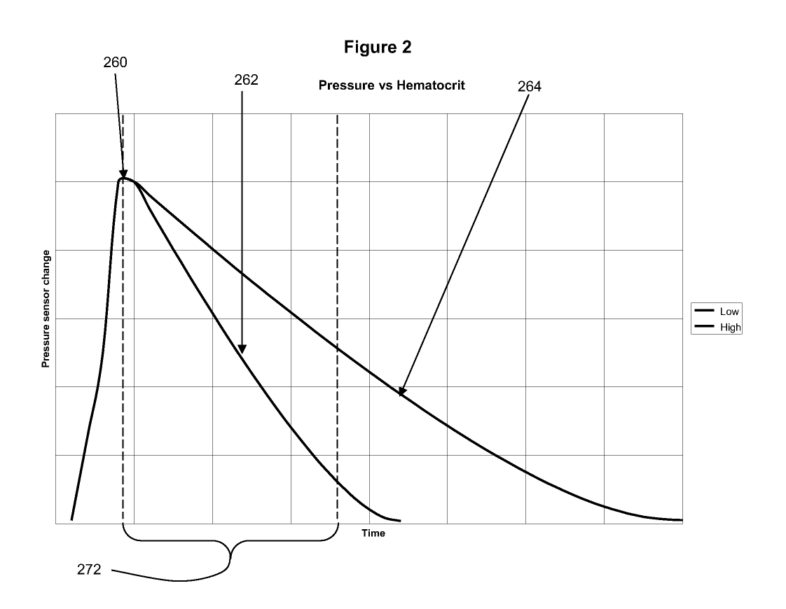

In another embodiment, a hematocrit or hematocrit related factor may estimated or determined based upon the time variation to relieve pressure through the dispensing assembly, which is believed to be dependent on the relative hematocrit level of the blood sample being withdrawn or provisioned. In another embodiment, a small internal diameter length of tubing or other type of flow restrictor may be inserted in the tubing path 111. The length and/or diameter of the restrictor may vary and need not be equal to the length of the tubing path 111 into which it is inserted. During the blood withdrawal and re-infusion cycle, the infusion and/or withdrawal resistance or pressure due the restrictor increases with increased hemoglobin concentrations and/or with increased hematocrit levels. Referring to FIG. 2, a graph of a pressure wave form and relative hematocrit reading from an orifice of known size is depicted. When the dispensing assembly is opened to the fluid channels containing the blood of the patient, it has been demonstrated that the pressure-time curve of the blood will initially increase to a peak pressure 260 as the pressurized blood is filling the dispensing assembly. In some embodiments, the peak pressure may be in the range of about 2 mm Hg to about 30 mm Hg or more, sometimes about 5 mm Hg to about 25 mm Hg, and other times about 10 mm Hg to about 30 mm Hg. In some embodiments, the dispensing force may be adjusted to achieve a particular peak pressure, with or without optional limits on the dispensing force and/or dispensed volume. Once the desired volume of blood has been provided to the dispensing assembly and the fluid communication with the fluid channels of the system have been closed, the fluid pressures in the dispensing assembly will begin to decline, but at different rates relating to the hematocrit, or blood viscosity and/or blood density, which are associated with the hematocrit. A lower hematocrit will descend at a faster rate (line 262) than a higher hematocrit (line 264). In the graph presented in FIG. 2, the low hematocrit data was based upon a hematocrit of 10%, while the higher hematocrit was based upon a hematocrit of 65%, but the actual results obtained may vary depending upon the sampling methology, sampling error, patient selection criteria, concomitant disease, the dimensions of the fluid channel, line pressurization, and other factors, for example. In some embodiments, the orifice may have a diameter or transverse dimension in the range of about 0.5 mm to about 2 mm, 1.2 mm to about 3.2 mm, and other times about 1 mm to about 1.6 mm. In some embodiments, the blood volume dispensed to generate the pressure waveform is in the range of about 50 μL to about 500 μL or more, other times about 25 μL to about 250 μL, and other times about 100 μL to about 300 μL. In some embodiments, the blood monitoring system provides a user with an indication of the relative change in a blood hematocrit level over time. In some embodiments, an absolute hematocrit reading at the beginning of the dispensing cycle may be used to calibrate the relative readings and therefore enables continuous hematocrit readings across a plurality of dispensing cycles to be achieved.

In some embodiments, a signal processor may be used to determine the relative hematocrit measurements based upon the average decay slopes of lines 262 and 264, while in other embodiments the area-under the curve may be calculated. In some embodiments, to improve the accuracy and/or reliability of the measurements a subset of lines 262 and 264 may be used. For example, in some embodiments, only the pressure measurement taken between the range of about 25% to about 75% of the curve from the peak pressure 260 to the zero pressure 268 and 270 may be used for the calculation. In another embodiment, for example, the pressure measurement at the 50% point between the peak pressure 260 to the zero pressure 268 and 270 is used. In still another embodiment, the hematocrit-related measurement may be based upon the pressure value at a certain time frame 272 after the peak pressure 260. Any of a variety of other suitable calculations may be used, however, to assess the hematocrit-related measurements. In another embodiment, the peak pressure 260, or the area under the curve between the onset 266 of the pressure increase and the peak pressure, may be assessed to determine the validity of the hematocrit-related measurement.

In another embodiment, the pressure response or waveform of blood to volume changes may be assessed using drawing or subatmospheric pressures to determine the hematocrit or other mechanical properties of blood. For example, FIG. 3 represents a graph of various pressure waveforms and their associated hematocrit readings of blood in response to a 150 cc/min draw flow rate through a flow restrictor comprising a tube having a fixed internal diameter of about 0.05 inches and a length of about 60 inches. During the draw phase, the slope lines 270, 272 and 274 of the pressure waveforms have been shown to vary with the total hemoglobin levels and/or hematocrit levels. The pressure drop, as measured between the pump and the flow restrictor, may be in the range of about −60 mm Hg to about −400 mm Hg or lower, but in some embodiments, the draw forces or draw flow rate may be reduced limit the magnitude of the negative pressure change. The draw volume may be in the range of about 1 cc to about 10 cc or more, sometimes about 2 cc to about 8 cc, and other times about 2.3 cc to about 7 cc. In some embodiments, limits may be placed on the negative pressure change to reduce the risk of cell lysis or draw attempts against a clogged fluid channel, for example. In one embodiment, the blood monitoring system may be configured with a draw pressure limit in the range of about −100 mm Hg to about −200 mm Hg, or sometimes about −150 mm Hg to about −166 mm Hg. In some embodiments, the tubing path 111 containing the restrictor may be provided in parallel with the other tubing of the system and may be selectively used as desired using one or more selection valves (not shown). In some embodiments, the hemoglobin, hematocrit or other blood parameter may be adjusted based upon the compliance of the material(s) comprising tubing path 111. The adjustment may be based, for example, on a slope adjustment to slope lines 270, 272 and 274, or the degree of deviation from a baseline waveform.

The various measurement procedures described herein may be used alone or in conjunction with each other or other assessment procedures to generate or estimate the hematocrit level of the blood, or to generate a correction factor that may be used to adjust the measurements of other blood tests that may be affected by the hematocrit or other hematocrit related factors, such as blood viscosity that was previously mentioned. Examples of blood tests that may benefit from adjustments relating to hematocrit include but are not limited to blood glucose and blood lactate.

In some embodiments, the hematocrit or correction factor may be used to adjust the volume of the blood sample dispensed by the sample dispenser. In some embodiments, the droplet morphology and/or the separation or transfer characteristics of the blood sample may vary with the hematocrit. For example, in some embodiments, variations in the hematocrit may cause variations in the percent of residual blood sample retained by the fluid dispenser. The relationship between the hematocrit and the volume adjustment may be linear or non-linear. By adjusting the volume of the blood sample, the accuracy and/or reliability of the final blood sample volume delivered to the test substrate may be improved. In some embodiments, the base volume of the blood sample may be adjusted up to about ±50% or more, sometimes up to about +25%, and other times up to about ±5% or about ±10%.

The above examples are merely illustrative of the many applications of the methods and systems of present invention. Although only a few embodiments of the present invention have been described herein, it should be understood that the present invention may be embodied in many other specific forms without departing from the spirit or scope of the invention. Therefore, the present examples and embodiments are to be considered as illustrative and not restrictive, and the invention may be modified within the scope of the appended claims.

What is claimed as new and desired to be protected by Letters Patent of the United States is:

1. A system for assessing a blood parameter, comprising:
a fluid channel;
a sensor system configured to detect a beginning and an end of a blood/non-blood interface in the fluid channel; and
a sensor processor configured to determine a difference factor between the beginning and the end of the blood interface.

2. The system of claim 1, wherein the sensor system comprises at least one optical sensor.

3. The system of claim 1, wherein at least one optical sensor is a movable optical sensor.

4. The system of claim 1, wherein the difference factor is a time-based difference factor.

5. The system of claim 1, wherein the difference factor is a distance-based difference factor.

6. The system of claim 1, wherein the sensor processor is further configured to generate a blood parameter using the difference factor.

7. The system of claim 6, wherein the blood parameter is selected from a hematocrit, a hemoglobin, a blood viscosity, and a blood density.

8. The system of claim 1, wherein the fluid channel is a tubing line.

9. The system of claim 1, further comprising:
a test medium advancement mechanism comprising a motor;
a blood sample dispenser; and
a fluid pump.

10. The system of claim 9, further comprising a vascular access device attachable to the fluid channel.

11. The system of claim 10, further comprising a plurality of test substrates.

12. A system for assessing a blood parameter, comprising:
a flow structure;
a sensor system configured to detect a pressure waveform of blood passing through the flow structure; and
a sensor processor configured to generate a blood parameter based upon the pressure waveform and the quantity of blood associated with the waveform.

13. The system of claim 12, wherein the flow structure is a flow restrictor.

14. The system of claim 12, wherein the flow structure is an open orifice.

15. The system of claim 12, wherein the blood parameter is selected from a hematocrit, a hemoglobin, a blood viscosity, and a blood density.

16. The system of claim 12, further comprising:
a test medium advancement mechanism comprising a motor;
a blood sample dispenser; and
a fluid pump.

17. The system of claim 16, further comprising a vascular access device attachable to the fluid channel.

18. The system of claim 16, further comprising a plurality test mediums.

19. A method for assessing a blood characteristic, comprising:
passing a volume of blood through a flow channel comprising having at least one flow channel structural characteristic;
measuring the pressure waveform of the volume of blood; and
determining a fluid characteristic of the volume of blood based upon the pressure waveform.

20. The method of claim 19, wherein determining the fluid characteristic of the volume of blood is further based upon at least one flow channel structural characteristic of the flow channel.

21. The method of claim 19, wherein the flow channel comprises a flow restrictor.

22. The method of claim 19, wherein the flow channel comprises an open orifice.

23. A method for assessing a blood characteristic, comprising:
withdrawing a blood sample from a patient and into a channel filled with a fluid;
assessing an interface between the blood sample and the fluid; and
generating a fluid characteristic based upon the interface.

24. The method of claim 19, wherein the interface between the fluid and the blood sample is selected from a group consisting of a temperature difference, a surface-to-surface interface and an optical difference.

25. The method of claim 19, wherein assessing the interface between the blood sample and the fluid comprises:
identifying a beginning interface point between the blood sample and the fluid; and
identifying an ending interface point between the blood sample and the fluid.

26. The method of claim 19, wherein generating the fluid characteristic based upon the interface comprises generating the fluid characteristic based upon a difference between the beginning interface and the ending interface.

27. The method of claim 26, wherein the difference is a time-based difference or a distance-based difference.

28. A method for performing blood monitoring, comprising:
obtaining a blood sample from a patient using an automated blood sampling assembly;
determining a non-reactive parameter and a reactive parameter of the blood sample; and
adjusting the reactive parameter based upon the non-reactive parameter parameter.

29. The method as in claim 28, further comprising returning at least a portion of the blood sample to the patient using the automated blood sampling assembly.

30. The method as in claim 28, wherein the non-reactive parameter is a hematocrit.

31. The method as in claim 28, wherein the non-reactive parameter is a mechanical parameter.

32. The method as in claim 28, wherein the mechanical parameter is blood viscosity or blood density.
The method as in claim 28, wherein the reactive parameter is blood glucose.

33. A method for performing blood monitoring, comprising:
obtaining a blood sample from a patient using an automated blood sampling assembly;
assessing a mechanical property of the blood sample; and
adjusting the automated blood sampling assembly based upon the mechanical property.

34. The method of claim 33, further comprising converting the mechanical property into a hematocrit-related measurement.

35. The method of claim 33, wherein adjusting the automated blood sampling assembly comprises setting a test substrate adjustment factor.

36. The method of claim 33, wherein adjusting the automated blood sampling assembly comprises setting a blood sample dispensing volume.
